(19) United States
(12) Patent Application Publication
(10) Pub. No.: US 2009/0211586 A1
(43) Pub. Date: Aug. 27, 2009

(54) DISPOSABLE COVER FOR CONTACT-TYPE LENS PIECE

(75) Inventors: William Shea, Pleasanton, CA (US); Keith Mullowney, Alamo, CA (US); Barry Linder, Danville, CA (US); Phil Baker, Walnut Grove, CA (US); Shelly Reed, Dublin, CA (US); Willem Crone, Oroville, CA (US); Yeou-Yen Cheng, Saratoga, CA (US)

Correspondence Address: LAW OFFICE OF CHARLES E. KRUEGER, P.O. BOX 5607, WALNUT CREEK, CA, 94596-1607, US

(73) Assignee: CLARITY MEDICAL SYSTEMS, INC., Pleasanton, CA (US)

(21) Appl. No.: 12/391,218

(22) Filed: Feb. 23, 2009

Related U.S. Application Data
(60) Provisional application No. 61/030,798, filed on Feb. 22, 2008.

(51) Int. Cl. A61B 19/00

(52) U.S. Cl. 128/849

(57) ABSTRACT

In one embodiment, a pliable film adapted to conform to parts of an optical examination device and provide a barrier between the eye of a patient being examined and the optical examination device.

### RELATED APPLICATIONS

This application claims priority from a provisional patent application entitled DISPOSAL COVER, Application No. 61/030,798 filed Feb. 22, 2008 which hereby is incorporated by reference for all purposes.

### TECHNICAL FIELD

The present invention relates to medical optical imaging, and in particular, to a disposable optical coupling element and the associated means of “snapping” the element to a contact imaging device in a sterile way such that it can act as a “sterile” barrier for the contact optical imaging device.

### BACKGROUND OF THE INVENTION

Various non-contact ophthalmic imaging devices exist but they all have limited angular field of view. The RetCam imaging system is a contact ophthalmic imaging device that can provide relatively large angular field of view for both eye fundus and anterior segment imaging. In the past, an optical coupling gel (such as the GenTeal gel) has been used as a bridging medium to accomplish the optical coupling function. The disadvantage is that after each use, there is a constant need for cleaning/disinfecting the contact portion of the multi-use imaging device before every use.

### BRIEF DESCRIPTION OF THE DRAWINGS

FIG. 1 illustrates an example embodiment of a disposable lens cover;

FIG. 2 illustrates a perspective view of an example embodiment of the disposable lens cover attached to a lens piece;

FIG. 3 illustrates a cross-sectional view of an example embodiment of the disposable lens cover attached to a lens piece;

FIG. 4 illustrates a cross-sectional view of an example embodiment of the disposable lens cover attached to a lens piece as well as being in contact with an eye;

FIGS. 5A-C depict an example embodiment of mounting the disposable lens cover onto the lens piece with the help of a disposable lens cover delivery system;

FIG. 6 depicts an example embodiment of molding only the optical coupling section so that it has a form similar to that of a contact lens to fit the radius of the eye. Further, the interior surface, which contacts the lens piece, is molded to intimately fit the device without the mounting section, as a result, it would “snap” over the tip of the lens piece and be ready for immediate use; and

FIG. 7 depicts an example embodiment of the disposable lens cover with the patient contact side molded to adjust/accommodate different ophthalmic applications such as anterior angle imaging, in which case, the optical coupling section is in the form of a wedge lens.

### DESCRIPTION OF EXAMPLE EMBODIMENTS

### Overview

The present disclosure described example embodiments that create a “sterile” barrier between a permanent, multi-use imaging device and the patient, while being optically “transparent” to the illumination and imaging path. In doing so, the reusable part has less of a burden or reliance on the user for disinfection of the multi-use device before every use, while not compromising image quality or usability. In one version, the index of refraction is chosen to optimize optical performance.

This is a new apparatus and the associated method that will allow end users to mount the “sterile” disposable barrier in a true “sterile” manner, quickly image a patient, reduce the risk of contamination due to inadequate or absent disinfection, and lessen the stress of long-term cleaning of the multi-use device. In addition, the device enables imaging in adults while in an upright position.

### Description

Reference will now be made in detail to various embodiments of the invention. Examples of these embodiments are illustrated in the accompanying drawings. While the invention will be described in conjunction with these embodiments, it will be understood that it is not intended to limit the invention to any embodiment. On the contrary, it is intended to cover alternatives, modifications, and equivalents as may be included within the spirit and scope of the invention as defined by the appended claims. In the following description, numerous specific details are set forth in order to provide a thorough understanding of the various embodiments. However, the present invention may be practiced without some or all of these specific details. In other instances, well known process operations have not been described in detail in order not to unnecessarily obscure the present invention. Further, each appearance of the phrase an “example embodiment” at various places in the specification does not necessarily refer to the same example embodiment.

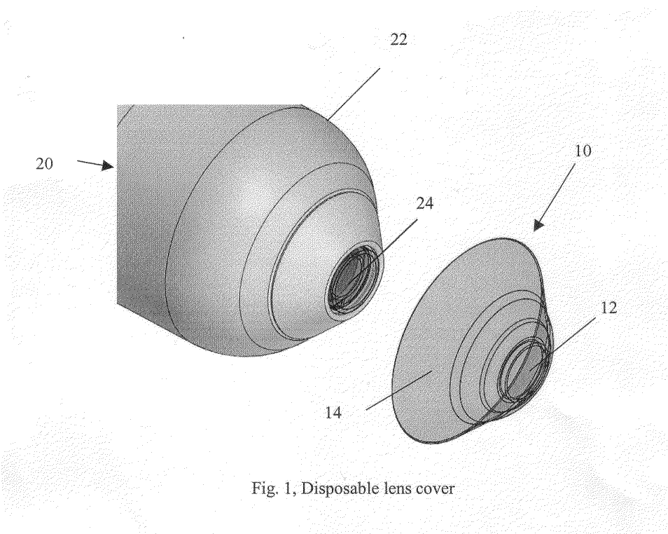

FIG. 1 depicts an example embodiment of the disposable “barrier” optical element, a lens cover 10. The disposable lens cover 10 includes a center optical coupling section 12 and a conical mounting section 14. Also depicted in FIG. 1 is a typical lens piece 20 of the RetCam® imaging system having a housing 22 and an optical aperture 24 in the form of a contact lens surface recessed from the housing 22.

Generally, an example embodiment of the disposable lens cover 10 is a flexible material or medium that covers the entire device-to-patent contact area and that may extend beyond the contact area. The material or medium may have enough pliability to conform to the patient contact area (eye) as well as the contour of the device (camera lens), or may have a preformed contour to fit to both the device and the patient contact area.

In one example embodiment the disposable lens cover 10 is constructed from two different materials. The optical coupling section 12 is constructed from a pliable material (such as a thin film analogous to Saran Wrap) thereby providing good flexibility at the point of contact with a patient's eye.

The conical mounting section 14 is constructed from a more rigid material than the optical coupling section 12 and provides rigidity beyond the point of contact. The conical mounting section 14 can also be formed to have a shape complementary to the shape of a particular lens piece housing to provide secure mounting. In an example embodiment the conical mounting section is preformed (molded) to “snugly” fit to the housing 22 of the lens piece 20, thus providing a mechanism to hold the disposable lens cover in place during use.

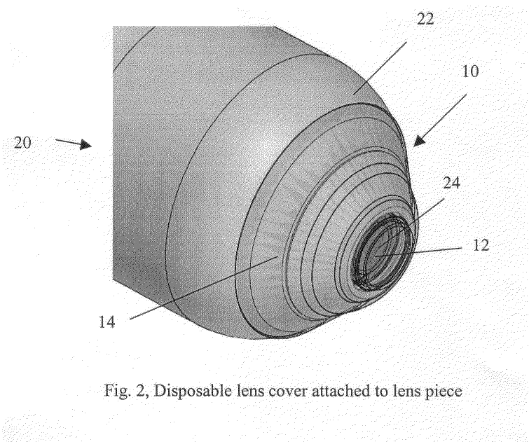

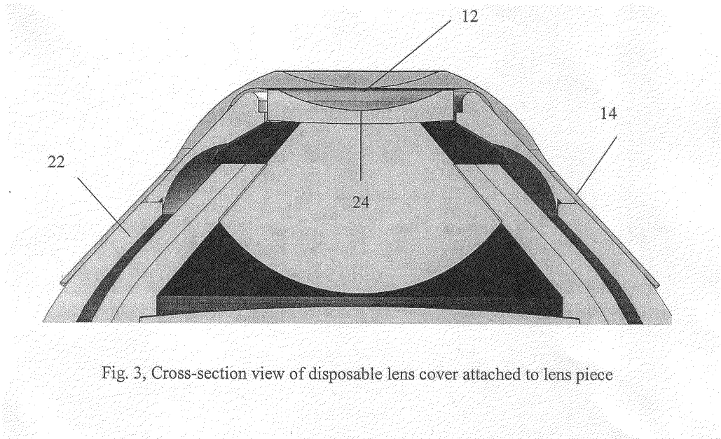

As depicted in FIGS. 2 and 3 in this example embodiment, the disposable lens cover is slipped directly over the lens piece housing. In another example embodiment the disposable lens cover is placed directly on the patient-to-device contact point.

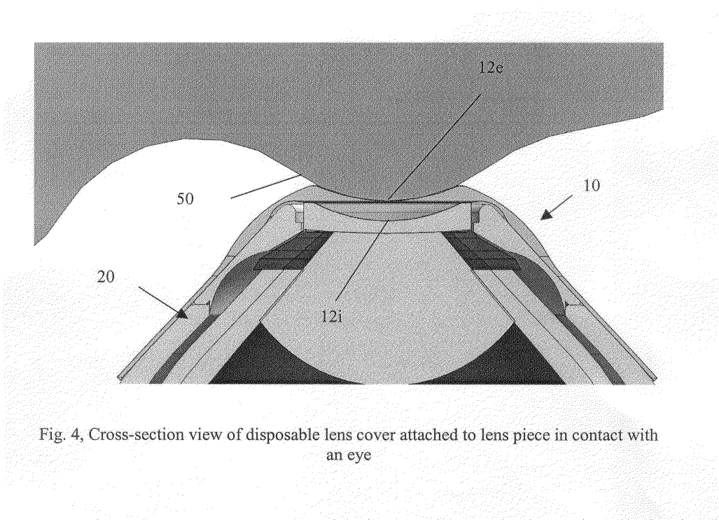

As depicted in FIG. 4, the optical coupling section 12 has an interior side 12i which abuts the lens piece 20 and an exterior side 12e which abuts the eye cornea surface 50 of the patient. In an example embodiment, an optically acceptable gel (such as GenTeal® gel manufactured by Novartis Pharmaceuticals Corporation) is placed between interior surface 12i of the optical coupling section 12 and lens piece 20. In this example embodiment, the optical gel also provides a “gluing” effect in keeping the barrier on device. An optical gel can also be applied to the exterior side 12e of the optical coupling section 12 to form a comfortable interface with patient's cornea 50.

The optical coupling section 12 may be made from hydrogel, glass, or medical grade “plastic”, or other materials used within the eye industry for contact applications. If the use of the disposable lens cover is not related to direct contact of the eye, then a broader set of materials can be deployed.

In another example embodiment, the disposable lens cover is designed to facilitate an easy method of individual dispensing, similar to a disposable cup dispenser. A potential side-effect of this easy use could be that the subsequent disposable lens cover (the next one to be pulled) might maintain its sterility; i.e. the exposed one protects the next, the next protects the 3rd, and so on. The devices could also be dispensed in individual sterile containers/carriers.

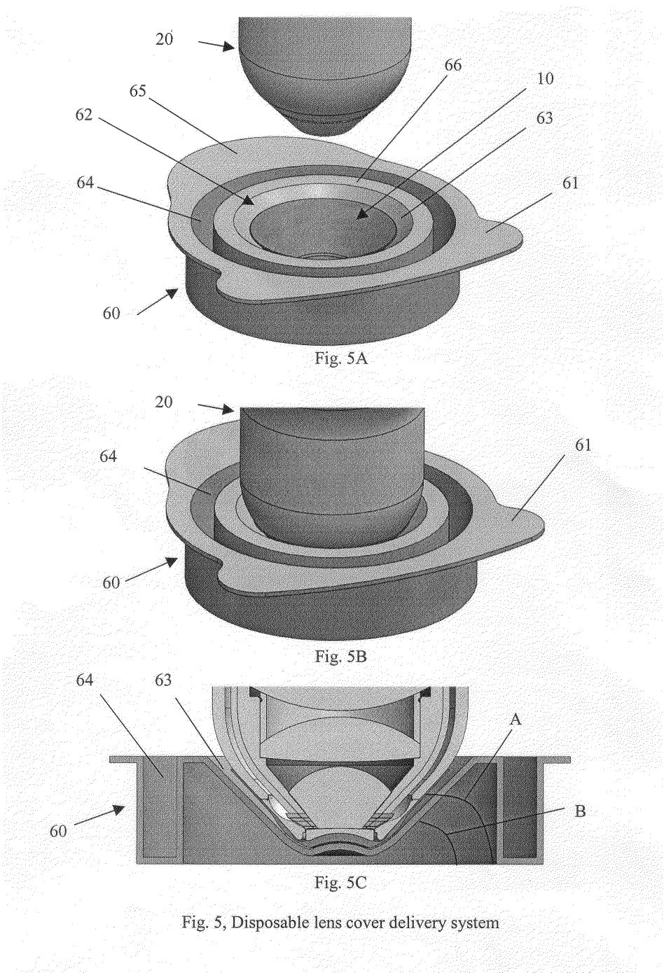

FIGS. 5A-C depict an example embodiment of an apparatus configured to mount the disposable lens cover 10 onto the lens piece 20 in a true “sterile” way with the help of a disposable lens cover delivery system 60 that includes a case 61 and a delivery system container 62 having an upper supporting surface 63 that holds the disposable lens cover 10. A reservoir 64 is formed between the case 61 and container 62.

The delivery system 60 is conceptually similar to a contact lens blister pack, but with a number of unique design features. The delivery system 60 functions in an intuitive manner, without the use of any additional tools or accessories. The delivery system tab 65 is held by end user's hand while the delivery system cover (not shown) is removed. The delivery system cover (not shown) is sealed along the container surface 66. Sterile liquid fills the container 62 that holds the disposable lens cover 10. The surrounding reservoir 64 is empty. In operation, the lens piece 20, which is further attached to a handpiece (not shown), is gently inserted into the delivery system container 62 that holds the disposable lens cover 10. The disposable lens cover 10 and the lens piece 20 are configured to mate to one another like a glove and hand. As the lens piece 20 is brought close to the disposable lens cover 10, the liquid that covers the disposable lens cover 10 is displaced into the surrounding reservoir 64 in order to maintain a clean and dry work area.

Once the camera lens piece 20 makes contact with the disposable lens cover 10, the delivery system container 62 is designed such that the suction between the lens cover 10 and lens piece 20 is greater than the suction between the lens cover 10 and the delivery system container 62. The surface finish of the delivery system container 62 minimizes the suction between the lens cover 10 and delivery system container 62. In example embodiments this reduction of suction is achieved by using a rough or wavy supporting surface 63 on the delivery container 62 or using a supporting surface 63 having raised structures.

Further, in other example embodiments the sides of the supporting surface 61 of the delivery system container 62 slant at a smaller angle than the slant of the part of the housing adjacent the aperture to minimizes the suction between the lens cover 10 and delivery system container 62. In this way, the disposable lens cover 10 transfers from the delivery system 60 to the camera lens piece 20.

As depicted in FIG. 5C, the housing slants out from the aperture at a first angle A measured from the horizontal. The supporting surface 63 of the container 62 slants at a second angle B, measured from the horizontal, where angle B is less than angle A thereby creating a space between the housing and the sides of the supporting surface 61 to reduce suction.

The lens piece 20 is then lifted away from the delivery system 60, and the disposable lens cover 10 adheres firmly to the lens piece 20 via suction while maintaining its sterility. The lens piece 20 and disposable lens cover 10 are then ready for use for imaging on a patient.

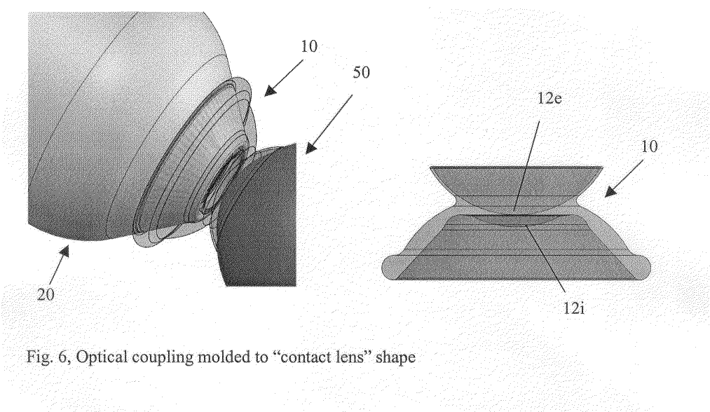

In another example embodiment, as depicted in FIG. 6, the optical coupling section 12 is molded to have a form similar to that of a contact lens, with the exterior surface 12e, which is in contact with eye 50 of the patient, molded to fit the radius of the eye 50. Further, the interior surface 12i, which contacts the lens piece 20, is molded to intimately fit the lens piece device 20. This would be one integrated component, device side and patient side. This example embodiment would not necessarily include a mounting section, instead it would “snap” over the tip of the lens piece and be ready for immediate use.

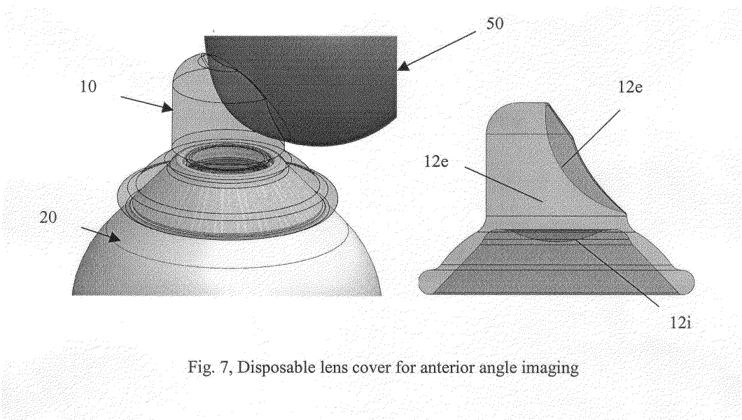

In another example embodiment, as depicted in FIG. 7, the patient contact side 12e is molded to adjust/accommodate any number of specific eye radii or different ophthalmic applications such as anterior angle imaging, for example. In these embodiments the optical coupling section 12 can be formed to have an indentation shaped to conform to the cornea of the eye of the patient. The location and orientation of the indentation can be varied to perform different functions, such as, for example, allowing the patient to sit or stand upright during an examination.

In this example embodiment, the optical coupling section can additionally or alternatively act as an optical element such as a lens or wedge for light beam forming or manipulation purposes. In particular, for anterior angle imaging, the optical coupling section 12 can be in the form of a wedge lens that is made of molded plastic, hydrogel, or a combination of materials.

The various example embodiments described may be designed/manufactured to contain optically acceptable gel (GenTeal®, for example) on either the device and or patient contact side. Thus maximizing optical coupling and lessen the design constraint of ideally matching surface-to-surface contact.

The disposable lens cover could be a flexible optical coupling disc that is pliable. In one example embodiment, the disc is in the form of a bag that contains an encapsulated optical gel or liquid that would deform to match both the device surface and the patient cornea, thus providing an optically transparent coupling.

Additionally, other example embodiments incorporate additional features that would aid in user manipulation or stability. For example, a finger “ring” could be integrated into or attached to the cover; a nose-bridge rest could be included; an attachment to “rest” on the forehead.

In another example embodiment, the optical coupling section includes a filter, such as a traditional “glass” filter integrated into the barrier or a “dye” included within the molding material that forms the optical coupling section that is coincident with the illumination annular ring beam (or return path) to discriminate particular wavelengths of interest. For example, the filter can be used for fluorescence angiography, imaging of fluorescein stained cornea, or red-free imaging.

In another example embodiment, the optical coupling section includes marks (etchings) coincident with the imaging path (or illumination path) to provide either dimensional calibration “tools” or dimensional references for alignment to image, measurements, and references to registration within the anatomy.

The optical coupling section can be formed to contain other lens materials or structures for specially designed imaging systems that allow specific optical designs used within the hydrogel or silicone matrix for a myriad of optical imaging or illumination functions.

The disposable lens cover can be used in any eye-contact ophthalmic imaging device, for example RetCam® camera, tonometer, pachymeter, and ultrasound. It could also be used, with possibly some modifications, for sonigraphs probes/contacts or tympanic measurements/probes. It can also be used to protect lens elements of a camera from debris, dust, and/or contamination. It can also be used for placement and/or removal of contact lenses. The device can also be applied onto a post-operative corneal surface to preclude surface irregularities for anterior or retinal examination. The device can also be applied to “Dipping Cone” lenses to conduct endothelial cell examinations or applied to anterior segment lenses of various designs for glaucoma angle evaluation.

The invention has now been described with reference to the example embodiments. Alternatives and substitutions will now be apparent to persons of skill in the art. Accordingly, it is not intended to limit the invention except as provided by the appended claims.

What is claimed is:

1. A disposable lens cover comprising:
a flexible optical coupling section formed of a material that is medically approved for contact with eye and configured to cover the contact lens of a contact-type ophthalmic imaging device, with the contact-type ophthalmic imaging device having a housing, an aperture, and a contact lens configured to be optically coupled to the eye of a patient; and
a mounting section, surrounding the flexible optical coupling section, preformed to mold to the housing to securely position the optical coupling section over the contact lens of the contact-type ophthalmic imaging device.

2. The disposable lens cover of claim 1 where:
the mounting section is made of a less flexible material than the optical coupling section.

3. The disposable lens cover of claim 2 where:
the flexible optical coupling section and the mounting section are formed of different materials.

4. The disposable lens cover of claim 1 where:
the flexible optical coupling section has an interior surface configured to face the contact lens of the contact-type ophthalmic imaging device and an exterior surface configured to face the eye and where the flexible optical coupling section has a shape such that the exterior surface conforms to the cornea of the eye of a patient when the cornea abuts the exterior surface.

5. The disposable lens cover of claim 4 where:
the optical coupling section has a shape such that the interior surface intimately fits the contact lens of the contact-type ophthalmic imaging device.

6. The disposable lens cover of claim 4 where:
the optical coupling section is formed to have a form similar to that of a contact lens, with the exterior surface molded to fit the radius of the eye the patient.

7. The disposable lens cover of claim 4 where:
the optical coupling section has an indention shaped to conform to the shape of the cornea of the eye of a patient.

8. The disposable lens cover of claim 7 where:
the indention is oriented to allow examination of a patient in the vertical position.

9. The disposable lens cover of claim 1 where:
the optical coupling section contains an encapsulated optical gel configured to deform to match the surface of the contact lens of the contact-type ophthalmic imaging device and to the cornea.

10. The disposable lens cover of claim 1 where:
the optical coupling section contains a filter to discriminate particular wavelengths of interest.

11. The disposable lens cover claim 1 where:
the optical coupling section includes marks configured to provide dimensional references for alignment to image, measurements or references to registration with the anatomy when the marks are coincident with an illumination beam.

12. The disposable lens cover claim 1 where:
the optical coupling section is in the shape of a wedge lens for anterior angle imaging

13. A disposable lens cover comprising:
a flexible optical coupling disc formed of a material that is medically approved for contact with eye and configured to fit within an aperture in the housing of a contact-type ophthalmic imaging device having a contact lens disposed within the aperture, with the flexible optical coupling disc having an interior surface configured to face toward the contact lens and an exterior surface configured to face toward the eye of the patient, with the flexible optical coupling disc having a shape such that the interior surface intimately fits the contact lens of the contact-type ophthalmic imaging device.

14. The disposable lens cover of claim 13 with:
the flexible optical coupling disc having a shape such that the exterior surface conforms to the cornea of the eye of a patient when the cornea abuts the exterior surface.

15. The disposable lens cover of claim 13 where:
the optical coupling disc is in the form of a bag and contains an encapsulated optical gel or liquid configured to deform to match the surface of the contact lens of the contact-type ophthalmic imaging device and to the cornea.

16. The disposable lens cover of claim 13 where:
the optical coupling disc is formed to have a form similar to that of a contact lens, with the exterior surface molded to fit the radius of the eye the patient.

17. The disposable lens cover of claim 13 where:
the optical coupling disc has indention shaped to conform to the shape of the cornea of the eye of a patient.

18. The disposable lens cover of claim 17 where:
the indention is oriented to allow examination of a patient in the upright position.

19. The disposable disc cover of claim 13 where:
the optical coupling section contains a filter to discriminate particular wavelengths of interest.

20. The disposable disc of claim 13 where:
the optical coupling disc includes marks configured to provide dimensional references for alignment to image, measurements or references to registration with the anatomy when the marks are coincident with an illumination beam.

21. An apparatus comprising:
a casing having a cylindrical receptacle, with the cylindrical receptacle having an inner surface;
a cylindrical container having an outer surface and an upper supporting surface, with the cylindrical container disposed within the receptacle with a reservoir formed between the inner surface of the cylindrical receptacle and the outer surface of the cylindrical container and with the upper receiving surface configured to support a disposable lens cover for covering the contact lens of a contact-type ophthalmic imaging device, with the contact-type ophthalmic imaging device having a housing, an aperture, and a contact lens configured to be optically coupled to the eye of a patient, with a section of housing surrounding the aperture slanting away from the aperture at a first angle A and with sides of the upper supporting surface slanting at an angle B where the angle B is less than the angle A so that space is formed when the housing abuts the upper supporting surface to reduce suction between the housing and the upper surface when mounting a disposable cover on housing.

22. The apparatus of claim 21 where:
the cylindrical container has a rough or wavy supporting surface to reduce suction between the supporting surface and the disposable lens cover.

23. The apparatus of claim 21 where:
the supporting surface has structures formed thereon to reduce suction between the supporting surface and the disposable lens cover.
